(19) United States
(12) Patent Application Publication
(10) Pub. No.: US 2022/0323535 A1
(43) Pub. Date: Oct. 13, 2022

(54) HEALTH SUPPLEMENT

(71) Applicant: Core Essentials, Inc., Pasadena, CA (US)

(72) Inventors: Jia Yia Liu, Riverside, CA (US); Lisa Ma, Alhambra, CA (US)

(73) Assignee: Core Essentials, Inc., Pasadena, CA (US)

(21) Appl. No.: 17/714,553

(22) Filed: Apr. 6, 2022

Related U.S. Application Data
(60) Provisional application No. 63/171,740, filed on Apr. 7, 2021.

(51) Int. Cl. A61K 36/9066; A61K 31/198; A61K 31/122; A61K 36/87; A61K 36/82; A61K 36/185; A61K 31/352; A61K 38/48; A61K 36/068; A61K 31/164; A61K 31/07; A61K 31/51; A61K 31/525; A61K 31/455; A61K 31/4415; A61K 31/714; A61K 31/375; A61K 31/593; A61K 33/32; A61K 33/30; A61K 33/04

(52) U.S. Cl. CPC A61K 36/9066; A61K 31/198; A61K 31/122; A61K 36/87; A61K 36/82; A61K 36/185; A61K 31/352; A61K 38/4873; A61K 36/068; A61K 31/164; A61K 31/07; A61K 31/51; A61K 31/525; A61K 31/455; A61K 31/4415; A61K 31/714; A61K 31/375; A61K 31/593; A61K 33/32; A61K 33/30; A61K 33/04

(57) ABSTRACT

A multi-component health supplement for improving overall wellness and modulating the immune system is provided as a mixture of N-acetyl-L-cysteine (NAC), quercetin, bromelain, Cordyceps, turmeric extract (curcumin), palmitoylethanolamide (PEA), grape seed extract, green tea extract, elderberry extract, pomegranate extract, and astaxanthin, and optionally one or more vitamins and minerals. Various subcombinations for targeting cellular health, fatigue, oxidative degradation, and other specific health needs are also provided.

### CROSS REFERENCE TO RELATED APPLICATION

This application claims the benefit of U.S. provisional application No. 63/171,740, filed Apr. 7, 2021, the entire contents of which are incorporated herein by this reference.

### FIELD OF THE INVENTION

The invention relates to health supplements for improving overall health and modulating the immune system.

### BACKGROUND OF THE INVENTION

The human body, although remarkably robust, is challenged daily by a number of stressors, both internal and external, including inadequate nutrition, lack of exercise, lack of sleep, stress, exposure to pathogens and environmental pollutants in food, air, and water, and the inescapable course of aging. Most recently, the global COVID-19 pandemic of 2020-2021 has emerged as a dangerous and stressful challenge. A stressed immune system makes the body more susceptible to disease, inflammation, fatigue, oxidative degradation, and even cancer.

It is known that certain vitamins, minerals, and botanicals can improve overall health or target specific health needs, but the sheer number of health supplements available in the marketplace is overwhelming, and the task of determining which supplements to take, and in what dose, is daunting. In addition, even if a health professional provides guidance, few consumers are likely to embrace a regimen that calls for swallowing 10, 15, or even 20+ tablets a day. A need exists for an all-in-one health supplement capable of supporting the body's immune system, improving cellular health, and promoting overall wellness.

Proponents of health supplements have been loosely divided between a “natural product” group, which favors botanicals and other organic components, such as green tea, turmeric, elderberry, etc., and a second group, which favors NAC and other amino acids and derivatives. Because of their respective biases, the two groups do not seem to work together. For example, proponents of botanical and other organic extracts tend to dismiss or even denounce the use of non-natural “chemical” components. Similarly, the second group often dismisses as “unproven” the health benefits of natural products, even when there are centuries-old accounts of the effectiveness of such products. This is unfortunate, and powerful combinations of components from these two groups, capable of stimulating a favorable cellular response, have been overlooked. A need exists for health supplements that draw on the best components that these two groups have to offer.

### SUMMARY OF THE INVENTION

In a first aspect, the present invention provides a multi-component health supplement to modulate the human immune system, improve cellular health, and promote overall wellness. In one embodiment, the supplement contains N-acetyl-L-cysteine (NAC), quercetin, bromelain, Cordyceps, turmeric extract (curcumin), palmitoylethanolamide, grape seed extract, green tea extract, elderberry extract, pomegranate extract, and astaxanthin. In another embodiment, eight vitamins and three minerals (vitamin A, Vitamin, C, Vitamin D, thiamine, riboflavin, niacin, vitamin B6, vitamin B12, magnesium, zinc, and selenium) are also included. Although the supplement can be provided in various forms, in one embodiment it is formulated as a powder, keeping in mind optimal absorption, with one or more flavoring agents added. The powder can be mixed with water to form a drink.

In a second aspect of the invention, several subsets (subcombinations) of these components are provided to enable specific health needs to be targeted. The subcombinations contain health-boosting components from both the “natural product” group and the “synthetic” group.

### DETAILED DESCRIPTION

In a first aspect of the invention, a multi-component health supplement is provided. In one embodiment, the supplement comprises a mixture of N-acetyl-L-cysteine (NAC), quercetin, bromelain, Cordyceps, turmeric extract (curcumin), palmitoylethanolamide (PEA), grape seed extract, green tea extract, elderberry extract, pomegranate extract, and astaxanthin. In a second embodiment, the supplement further comprises at least one vitamin or mineral selected from the group consisting of vitamin A, vitamin B1 (thiamine), vitamin B2 (riboflavin), vitamin B3 (niacin), vitamin B6 (pyridoxine), vitamin B12 (cobalamin), vitamin C, vitamin D, magnesium, zinc, and selenium. In a third embodiment, the supplement comprises a mixture of NAC, quercetin, bromelain, Cordyceps, turmeric extract, PEA, grape seed extract, green tea extract, elderberry extract, pomegranate extract, astaxanthin, vitamin A, vitamin B1, vitamin B2, vitamin B3, vitamin B6, vitamin B12, vitamin C, vitamin D, magnesium, zinc, and selenium. Each component serves one or more functions, and collectively the components act synergistically to boost the immune system, improve cellular health, reduce oxidative degradation, and enhance overall wellness.

N-acetyl-L-cysteine (NAC) is the N-acetyl derivative of the amino acid L-cysteine. It is a precursor to the antioxidant glutathione, which promotes cellular health in the human body.

Quercetin is a plant flavonoid found in many fruits and vegetables. An antioxidant, quercetin promotes immune health. As used herein, the term “quercetin” includes any of quercetin aglycone, quercetin dihydrate, isoquercetin, and mixtures thereof.

Turmeric Extract (Curcoma longa) contains curcumin. It optimizes the immune system for vitality and endurance.

Elderberry Extract (Sambucus nigra L.) optimizes immunity in respiratory viral infections.

Cordyceps is a fungus related to the mushroom. It optimizes the immune system for vitality and endurance. Examples of Cordyceps include the fruiting body of Cordyceps militaris and Cordyceps sinensis, Cs-4 (fermented Cordyceps), and extracts thereof.

Grape Seed Extract (Vitis Vinifera) contains proanthocyanidins, a class of polyphenols that enhance circulation and immunity.

Green Tea Extract (Camelia sinesis leaf) is rich in catechins such as epigallocatechin gallate (EGCG). It reduces inflammation and promotes cellular health.

Palmitoylethanolamide (PEA) is a fatty acid amide. It engages endocannabinoid receptors to provide a calming effect and mood enhancement.

Astaxanthin (Haematococcus pluvialis) whole algae is beneficial for the skin, heart, and circulation.

Bromelain (Ananas comosus) is an enzyme extract derived from the stem of pineapples. It is rich in antioxidants to promote the immune system.

Pomegranate Extract (Punica granatum L. rind) is rich in antioxidants to promote the immune system.

The botanicals and other natural components referred to herein can be provided as extracts (e.g., aqueous extracts, alcoholic extracts, etc.), powders, or other forms derived from the seed, rind, fruiting body, or other part of the natural material in a manner familiar to the health supplement industry.

Table 1 presents broad, intermediate, and more narrow dose ranges of NAC, PEA, and other natural components, for a human adult, in one embodiment of the invention. Broad ranges roughly equate to dose per day, while narrow ranges more typically reflect dose/serving.

TABLE 1
Selected Component Dosages (in milligrams, mg)

| Component | Broad | Intermediate | Narrow |
|---|---|---|---|
| N-acetyl-L-cysteine | 40-1400 | 100-800 | 200-600 |
| Turmeric Extract (Curcoma Longa) | 50-2000 | 50-500 | 100-300 |
| Elderberry Extract (Sambucus nigra) | 50-1000 | 100-750 | 100-400 |
| Mushroom extract (Cordyceps militaris | 50-1000 | 100-500 | 100-300 |
| fruiting body) | | | |
| Grape seed extract (Vitis vinifera) | 50-600 | 50-400 | 50-200 |
| (proanthocyanidin) | | | |
| Quercetin | 20-1200 | 50-700 | 100-300 |
| Green Tea extract (Camelia sinesis | 5-1000 | 25-500 | 25-150 |
| leaf) (EGCG) | | | |
| Palmitoylethanolamide | 10-1200 | 20-600 | 20-400 |
| Astaxanthin (Haematococcus pluvialis) | 2-30 | 6-30 | 12-30 |
| whole algae | | | |
| Bromelain (Ananas comosus stem) | 10-850 | 10-500 | 10-250 |
| Pomegranate Extract (Punic gratum | 10-500 | 10-250 | 10-50 |
| L., rind) | | | |

In addition to NAC, PEA, and the natural (e.g., botanical) components, the multi-component supplement contains several vitamins and minerals. The skilled person will understand that, in the context of a particular vitamin, e.g., vitamin A, vitamin B, etc., there may be several closely related compounds and their physiologically compatible salts, esters, and related moieties. For example, “vitamin A” denotes any of retinol, a retinyl ester (e.g., retinyl palmitate), and a provitamin A carotenoid (e.g., beta-carotene). “Vitamin D” denotes any of vitamin D2 (ergocalciferol) and vitamin D3 (cholecalciferol). “Vitamin C” denotes ascorbic acid and/or its bioavailable salts (e.g., calcium ascorbate, dehydroascorbate, etc.). “Vitamin B3” denotes niacin (aka nicotinic acid), niacinamide (aka nicotinic acid amide or nicotinamide), or even inositol nicotinate. Certain vitamins can be provided as a hydrochloride or other physiologically compatible salt. Two examples are “thiamin” (aka thiamine), which can be provided as thiamin hydrochloride, and vitamin B6, which can be provided as pyridoxine hydrochloride. Vitamin B12 can exist in four nearly identical chemical forms or “vitamers,” i.e., cyanocobalamin, hydroxocobalamin, adenosylcobalamin and methylcobalamin. Regardless of the particular form, salt, or moiety, all such compounds are understood to be included within the scope of the term “vitamin” as used herein.

The skilled person will also understand that the “minerals” magnesium, selenium, and zinc are generally not provided in elemental form but, rather, in a bioavailable form commonly employed in the pharmaceutical, nutraceutical, and/or vitamin industries. Nonlimiting examples include magnesium citrate, glycinate, lactate, malate, taurate, and L-threonate; zinc gluconate, picolinate, citrate, acetate, glycerate, and monomethionine; and seleno-L-methionine and seleno-L-cysteine. As used herein, the terms “magnesium,” “selenium,” and “zinc” refer to a bioavailable form of the mineral.

Table 2 provides a nonlimiting example of a vitamin and mineral dose contained in a single serving (7.5 g on a dry weight basis) of a multi-component supplement according to one embodiment of the invention, wherein “mg” denotes milligrams, “mcg” denotes micrograms, and “IU” denotes International Units. “% Daily Values (DV)” are based on a 2000 calorie diet.

TABLE 2
Vitamins in a Single Serving

| | | | % Daily |
|---|---|---|---|
| Component | Amount | | Value |
| Vitamin A (as retinyl palmitate) | 525 | mcg | 58% |
| Vitamin C (as ascorbic acid) | 1000 | mg | 1111% |
| Vitamin D (as cholecalciferol) | 25 mcg (1000 IU) | | 125% |
| Thiamin (as thiamin hydrochloride) | 25 | mg | 2083% |
| Riboflavin | 25 | mg | 1923% |
| Niacin (as niacinamide) | 4 | mg | 25% |
| Vitamin B6 (as pyroxidine | 3 | mg | 176% |
| hydrochloride) | | | |
| Vitamin B12 (as methylcobalamin) | 10 | mcg | 417% |
| Magnesium (as magnesium bisglycinate | 100 | mg | 24% |
| chelate) | | | |
| Zinc (as zinc gluconate) | 10 | mg | 91% |
| Selenium (as seleno-L-methionine) | 18 | mcg | 33% |

The supplement can be provided in powder, liquid, tablet, or other form, such as the form of supplements encountered in the pharmaceutical and nutraceutical industries. In one embodiment a multi-component health supplement is prepared as a powder by mixing NAC, PEA, botanicals, vitamins, and minerals. Additional excipients (flavoring, processing aides, added sugar, etc.) may also be included during formulation. Solid components can first be reduced in particle size using (e.g.) trituration, pulverization by intervention, or levigation, and then combined with any liquid components, dried again as necessary, and then sieved to achieve a uniform particle size distribution. A very coarse powder has an average particle size greater than 1000 microns; coarse 355-1000; moderately coarse 180-355; fine 125-180; very fine 90-125. (As a point of reference, ordinary granulated sugar has fine crystals of around 400 microns in size, while powdered sugar has an average particle size of about 50 microns.) Depending on the exact composition of the supplement and the relative amounts of more water soluble and less water soluble components, powders of a particular average particle size may be appropriate. The powder can be packaged for sale (e.g., in a single-size serving, about 7.5 g) and can be added to water or another beverage and consumed as a liquid.

In another aspect of the invention, mixtures of various subcombinations of the components described herein are provided, in powdered or other form, typically in small quantities of a few grams or less. In powdered form, each subcombination can be mixed with water or another beverage and taken as a stand-alone supplement to target one or more particular health conditions or effects, including pain, mood, fatigue, memory, respiratory and cardiovascular health, exercise performance, endurance, joint health, gastrointestinal health, skin health, nerve support, respiratory health, vascular health, and hay fever. Although not bound by theory, it is believed that each of these subcombinations of components offer a synergistic effect greater than the sum of their individual components. They serve as both easy to take, stand-alone supplements and as platforms for further clinical studies. Nonlimiting examples are presented in Tables 3 and 4. In one embodiment, a health supplement contains NAC and astaxanthin (AST), or NAC, AST, and one or more additional components (see Table 3). In another embodiment, a health supplement contains NAC and palmitoylethanolamide (PEA), or NAC, PEA, and one or more additional components (see Table 4). Additional embodiments of health supplements containing multiple components can be prepared by combining the components of any of the examples listed in Tables 3 and 4 with one or more additional components from Tables 1 and/or 2.

TABLE 3
Multi-Component Supplements Based on NAC and AST

| | Function/health | | Component Dosage Range (mg) | | |
|---|---|---|---|---|---|
| No. | condition* | Components | Broad | Intermediate | Narrow |
| 1 | MS, M, RC, EP, | NAC | 40-1400 | 40-800 | 200-600 |
| | E | AST | 2-30 | 6-30 | 12-30 |
| 2 | MS, M, RC, EP, | NAC | 40-1400 | 40-800 | 200-600 |
| | E | AST | 2-30 | 6-30 | 12-30 |
| | | Grape Seed | 50-600 | 50-400 | 50-200 |
| | | Extract | | | |
| 3 | MS, M, RC, EP, | NAC | 40-1400 | 40-800 | 200-600 |
| | E, J, GI | AST | 2-30 | 6-30 | 12-30 |
| | | Turmeric | 50-2000 | 50-500 | 100-300 |
| | | Extract | | | |
| 4 | MS, M, RC, EP, | NAC | 40-1400 | 40-800 | 200-600 |
| | E | AST | 2-30 | 6-30 | 12-30 |
| | | Green tea | 5-1000 | 25-500 | 25-150 |
| | | Extract | | | |
| 5 | MS, M, RC, EP, | NAC | 40-1400 | 40-800 | 200-600 |
| | E | AST | 2-30 | 6-30 | 12-30 |
| | | Cordyceps | 50-1000 | 100-500 | 100-300 |
| 6 | RC, EP, E | NAC | 40-1400 | 40-800 | 200-600 |
| | | AST | 2-30 | 6-30 | 12-30 |
| | | Elderberry | 50-1000 | 100-750 | 100-400 |
| | | Extract | | | |
| 7 | MS, M, RC, EP, | NAC | 40-1400 | 40-800 | 200-600 |
| | E, S | AST | 2-30 | 6-30 | 12-30 |
| | | Quercetin | 20-1200 | 50-700 | 100-300 |
| 8 | MS, M, RC, EP, | NAC | 40-1400 | 40-800 | 200-600 |
| | E, S, GI | AST | 2-30 | 6-30 | 12-30 |
| | | Bromelain | 10-850 | 10-500 | 10-250 |
| 9 | MS, M, RC, EP, | NAC | 40-1400 | 40-800 | 200-600 |
| | E, S | AST | 2-30 | 6-30 | 12-30 |
| | | Pomegranate | 10-500 | 10-250 | 10-50 |
| | | Extract | | | |

*MS—mood stabilizer, M—memory support, RC—respiratory and cardiovascular support, EP—exercise performance, E—endurance, J—joint health, GI—gastrointestinal health, S—skin health, N—nerve support, R—respiratory health, V—vascular support, F—fatigue, P—pain, H—hay fever

TABLE 4
Multi-Component Supplements Based on NAC and PEA

| | Function/health | | Component Dosage Range (mg) | | |
|---|---|---|---|---|---|
| No. | condition* | Components | Broad | Intermediate | Narrow |
| 10 | P, M, MS, N, R | NAC | 40-1400 | 40-800 | 200-600 |
| | | PEA | 2-30 | 6-30 | 12-30 |
| 11 | P, M, MS, V, R | NAC | 40-1400 | 40-800 | 200-600 |
| | | PEA | 10-1200 | 20-600 | 20-400 |
| | | Grape Seed | 50-600 | 50-400 | 50-200 |
| | | Extract | | | |
| 12 | P, M, MS, J, V | NAC | 40-1400 | 40-800 | 200-600 |
| | | PEA | 10-1200 | 20-600 | 20-400 |
| | | Turmeric | 50-2000 | 50-500 | 100-300 |
| | | Extract | | | |
| 13 | P, M, MS, EP, | NAC | 40-1400 | 40-800 | 200-600 |
| | E, R | PEA | 10-1200 | 20-600 | 20-400 |
| | | Green tea | 5-1000 | 25-500 | 25-150 |
| | | Extract | | | |
| 14 | P, M, MS, EP, | NAC | 40-1400 | 40-800 | 200-600 |
| | E, F, R | PEA | 10-1200 | 20-600 | 20-400 |
| | | Cordyceps | 50-1000 | 100-500 | 100-300 |
| 15 | P, M, MS, R | NAC | 40-1400 | 40-800 | 200-600 |
| | | PEA | 10-1200 | 20-600 | 20-400 |
| | | Elderberry | 50-1000 | 100-750 | 100-400 |
| | | Extract | | | |
| 16 | P, M, MS, H, R | NAC | 40-1400 | 40-800 | 200-600 |
| | | PEA | 10-1200 | 20-600 | 20-400 |
| | | Quercetin | 20-1200 | 50-700 | 100-300 |
| 17 | P, M, MS, J, R, | NAC | 40-1400 | 40-800 | 200-600 |
| | GI, S | PEA | 10-1200 | 20-600 | 20-400 |
| | | Bromelain | 10-850 | 10-500 | 10-250 |
| 18 | P, M, MS, S, R | NAC | 40-1400 | 40-800 | 200-600 |
| | | PEA | 10-1200 | 20-600 | 20-400 |
| | | Pomegranate | 10-500 | 10-250 | 10-50 |
| | | Extract | | | |
| 19 | P, M, MS, J, R, | NAC | 40-1400 | 40-800 | 200-600 |
| | GI, S, EP, E | PEA | 2-30 | 6-30 | 12-30 |
| | | Bromelain | 10-850 | 10-500 | 10-250 |
| | | Cordyceps | 50-1000 | 100-500 | 100-300 |
| | | Turmeric | 50-2000 | 50-500 | 100-300 |
| | | Extract | | | |

*MS—mood stabilizer, M—memory support, RC—respiratory and cardiovascular support, EP—exercise performance, E—endurance, J—joint health, GI—gastrointestinal health, S—skin health, N—nerve support, R—respiratory health, V—vascular support, F—fatigue, P—pain, H—hay fever

Upon reading this disclosure, other embodiments and modifications within the scope of the present invention may be apparent to the skilled person. The invention is limited only by the appended claims and equivalents thereof.

What is claimed is:

1. A multi-component health supplement comprising:
a mixture of N-acetyl-L-cysteine (NAC), astaxanthin, and one or more components selected from the group consisting of grape seed extract, turmeric extract, green tea extract, Cordyceps, elderberry extract, quercetin, bromelain, and pomegranate extract.

2. The health supplement of claim 1, further comprising at least one vitamin or mineral selected from the group consisting of vitamin A, vitamin B1, vitamin B2, vitamin B3, vitamin B6, vitamin B12, vitamin C, vitamin D, magnesium, zinc, and selenium.

3. A multi-component health supplement comprising:
a mixture of N-acetyl-L-cysteine (NAC), palmitoylethanolamide (PEA), and one or more components selected from the group consisting of grape seed extract, turmeric extract, green tea extract, Cordyceps, elderberry extract, quercetin, bromelain, and pomegranate extract.

4. The health supplement of claim 3, further comprising at least one vitamin or mineral selected from the group consisting of vitamin A, vitamin B1, vitamin B2, vitamin B3, vitamin B6, vitamin B12, vitamin C, vitamin D, magnesium, zinc, and selenium.

5. A multi-component health supplement comprising:
a mixture of N-acetyl-L-cysteine (NAC), astaxanthin and/or palmitoylethanolamide (PEA), and one or more components selected from the group consisting of grape seed extract, turmeric extract, green tea extract, Cordyceps, elderberry extract, quercetin, bromelain, and pomegranate extract.

6. The health supplement of claim 5, wherein the mixture comprises NAC, PEA, bromelain, Cordyceps, and turmeric extract.

7. A multi-component health supplement, comprising:
a mixture of N-acetyl-L-cysteine (NAC), quercetin, bromelain, Cordyceps, turmeric extract, palmitoylethanolamide (PEA), grape seed extract, green tea extract, elderberry extract, pomegranate extract, and astaxanthin.

8. The health supplement of claim 5, further comprising vitamin A, vitamin B1, vitamin B2, vitamin B3, vitamin B6, vitamin B12, vitamin C, vitamin D, magnesium, zinc, and selenium.

9. A health supplement for boosting a human's immune system, comprising:
a mixture of NAC, PEA, zinc, astaxanthin, vitamin A, vitamin D, vitamin C, elderberry extract, green tea extract, turmeric extract, grape seed extract, and Cordyceps.

10. A health supplement for enhancing cellular health, comprising:
a mixture of NAC, PEA, magnesium, selenium, astaxanthin, thiamin, vitamin D, vitamin A, vitamin B6, vitamin B12, vitamin B3, quercetin, turmeric extract, grape seed extract, pomegranate extract, and Cordyceps.

11. An antioxidant health supplement, comprising:
a mixture of NAC, PEA, zinc, selenium, astaxanthin, vitamin B1, vitamin B2, vitamin A, vitamin C, elderberry extract, green tea extract, turmeric extract, and grape seed extract.
